(19) United States
(12) Patent Application Publication
(10) Pub. No.: US 2012/0083010 A1
(43) Pub. Date: Apr. 5, 2012

(54) METHOD FOR DIAGNOSING POMPE DISEASE

(76) Inventors: Hung V. Do, New Hope, PA (US); Brian E. Rane, Somerset, NJ (US); John Flanagan, East Windsor, NJ (US)

(21) Appl. No.: 13/212,917

(22) Filed: Aug. 18, 2011

Related U.S. Application Data
(63) Continuation of application No. PCT/US2010/024553, filed on Feb. 18, 2010.
(60) Provisional application No. 61/153,506, filed on Feb. 18, 2009.

(51) Int. Cl. C12Q 1/34

(52) U.S. Cl. 435/18

(57) ABSTRACT

Provided is a method for diagnosing Pompe disease in a patient by measuring acid α-glucosidase activity in a sample from the patient. The invention also provides a method for monitoring the treatment of Pompe disease with specific pharmacological chaperones by measuring acid α-glucosidase activity in a sample from the patient.

### CROSS-REFERENCE TO RELATED APPLICATION

The present application claims the benefit of U.S. Provisional Application No. 61/153,506, filed Feb. 18, 2009, which is hereby incorporated by reference in its entirety.

### FIELD OF THE INVENTION

The present invention provides a method for diagnosing an individual having Pompe disease by determining the level of acid α-glucosidase activity in a biological sample from the patient. The present invention also provides a method for monitoring the treatment of an individual having Pompe disease by determining the level of acid α-glucosidase activity in a biological sample from the patient during treatment for the disease.

### BACKGROUND

Pompe disease (acid maltase deficiency) is caused by a deficiency in the enzyme acid α-glucosidase (GAA). GAA metabolizes glycogen, a storage form of sugar used for energy, into glucose. The accumulation of glycogen leads to progressive muscle myopathy throughout the body which affects various body tissues, particularly the heart, skeletal muscles, liver, and nervous system. According to the National Institute of Neurological Disorders and Stroke, Pompe disease is estimated to occur in about 1 in 40,000 births.

There are three recognized types of Pompe disease—infantile, juvenile, and adult onset (see, e.g., Hirschhorn and Reuser, In: Scriver C R, Beaudet A L, Sly W, Valle D, editors; The Metabolic and Molecular Bases of Inherited Disease, Vol. III, New York: McGraw-Hill; 2001. p. 3389-420, 2001: 3389-3420). Infantile-onset Pompe Disease is the most severe, and presents with symptoms that include severe lack of muscle tone, weakness, enlarged liver and heart, and cardiomyopathy. Swallowing may become difficult and the tongue may protrude and become enlarged. Most children die from respiratory or cardiac complications before the age of two, although a sub-set of infantile-onset patients live longer (non-classical infantile patients). Juvenile onset Pompe disease first presents in early to late childhood and includes progressive weakness of the respiratory muscles in the trunk, diaphragm, and lower limbs, as well as exercise intolerance. Most juvenile onset Pompe patients do not live beyond the second or third decade of life. Adult onset symptoms involve generalized muscle weakness and wasting of respiratory muscles in the trunk, lower limbs, and diaphragm. Some adult patients are devoid of major symptoms or motor limitations.

Unless identified during pre-natal screening, diagnosis of Pompe disease is a challenge. Diagnosis of adult-onset Pompe is even more difficult since number, severity, and type of symptoms a patient experiences vary widely, and may suggest more common disorders such as muscular dystrophies. Diagnosis is confirmed by measuring α-glucosidase activity and/or detecting pathologic levels of glycogen from biological samples.

Pompe disease is one of several of glycogen pathologies. Others include Debrancher deficiency (Cori's-Forbes' disease; Glycogenosis type III); Branching deficiency (Glycogenosis type IV; Andersen's disease); Myophsophorylase (McArdle's disease, Glycogen storage disease V); Phosphofructokinase deficiency-M isoform (Tauri's disease; Glycogenosis type VII); Phosphorylase b Kinase deficiency (Glycogenosis type VIII); Phosphoglycerate kinase A-isoform deficiency (Glycogenosis IX); Phosphoglycerate M-mutase deficiency (Glycogenosis type X).

Pompe disease is often difficult to diagnose in part because GAA enzyme activity measurements in blood or mixed leukocyte samples (lymphocytes with up to 10% contaminating granulocytes) are unreliable using the traditional 4-methylumbelliferyl-α-D-glucopyranoside (4MU-α-Glc) fluorometric assay. Multiple enzymes, predominantly maltase-glucoamylase (MGAM), hydrolyze this substrate and mask the GAA enzyme deficiency. The addition of acarbose, an inhibitor of MGAM, has been shown to improve this assay but is still insufficient to distinguish small differences in GAA activity in mixed leukocyte samples. Thus, a need exists to reliably and specifically measure GAA enzyme activity in mixed leukocyte samples.

### SUMMARY OF THE INVENTION

One aspect of the present invention provides methods for diagnosing Pompe disease in a subject, which methods comprise determining the activity of acid α-glucosidase (GAA) in a biological sample from the subject. In one non-limiting embodiment, the method of determining the activity of acid α-glucosidase activity in the sample is an assay comprising parallel enzymatic reactions, wherein a first enzymatic reaction is conducted in the absence of an antibody to acid α-glucosidase (anti-GAA antibodies), and a second enzymatic reaction is conducted in the presence of an antibody to acid α-glucosidase wherein a difference between the two enzymatic assays indicates acid α-glucosidase activity.

In one embodiment, the method of determining the activity of acid α-glucosidase activity is an assay comprising an enzymatic reaction which measures the hydrolysis of a GAA substrate, for example, 4-methylumbelliferyl-α-D-glucopyranoside (4MU-α-Glc).

In another embodiment, the anti-GAA antibody inhibits GAA activity in the assay.

In one embodiment, the anti-GAA antibody is a polyclonal antibody.

In another embodiment, the anti-GAA antibody is a monoclonal antibody.

In another embodiment, the biological sample is peripherally obtained lymphoblasts, leukocytes or polymorphonuclear cells (PMNs), or mixtures thereof, derived from an individual.

In another embodiment, the sample is cultured fibroblasts derived from an individual.

In another embodiment, the method of determining the activity of acid α-glucosidase activity in a patient sample comprises the following steps:

(a) measuring acid α-glucosidase enzymatic activity of the sample in the absence of an anti-GAA antibody;

(b) measuring acid α-glucosidase enzymatic activity of the sample in the presence of an anti-GAA antibody;

(c) comparing the enzymatic activity of steps (a) and (b), wherein a difference in enzymatic activity between (a) and (b) is the acid α-glucosidase activity in the sample.

In one embodiment, the present invention provides a method for diagnosing Pompe disease in a patient, which method comprises detecting GAA activity in a first sample from a patient, and comparing the level of GAA activity to the level of GAA activity measured in a second sample from a healthy individual who does not have Pompe disease, wherein a lower level of GAA activity in the first sample compared to the second sample indicates Pompe disease.

The present invention also provides a method for monitoring treatment of a Pompe disease patient, which method comprises determining the activity of acid α-glucosidase in a sample from the patient, wherein an increase in GAA activity following treatment indicates that the individual is responding to the treatment for the disease.

In one embodiment of the invention, the subject is administered a specific pharmacological chaperone for acid α-glucosidase upon receiving a positive result from any one of the above-described methods and assays. In a further embodiment, the specific pharmacological chaperone used in the therapy is an inhibitor of GAA, such as a reversible competitive inhibitor. In one specific embodiment, the inhibitor is 1-deoxynojirimycin (DNJ), or a pharmaceutically acceptable salt thereof.

In other embodiments, the patient is treated with enzyme replacement therapy (ERT) upon receiving a positive result from any one of the above-described methods and assays. GAA may be obtained from commercial sources or may be obtained by synthesis techniques known to a person of ordinary skill in the art. The wild-type enzyme can be purified from a recombinant cellular expression system, human placenta, animal milk, or plants.

In one embodiment, the GAA is a recombinant human GAA (rhGAA), for example, GAA is alglucosidase alfa, which consists of the human enzyme acid α-glucosidase (GAA), encoded by the most predominant of nine observed haplotypes of this gene and is produced by recombinant DNA technology in a Chinese hamster ovary cell line. Alglucosidase alpha is available as Myozyme®, from Genzyme Corporation (Cambridge, Mass.).

In one embodiment, a subject is administered a combination of a specific pharmacological chaperone (e.g. a reversible competitive inhibitor (e.g. 1-DNJ)) and ERT (e.g. a recombinant GAA, rhGAA, (e.g., Myozyme®, Genzyme Corp., Cambridge, Mass.)).

The present invention also provides a method for treating Pompe disease with an effective amount of a specific pharmacological chaperone that reversibly binds to the GAA, and monitoring its effect on GAA activity, wherein an increase in GAA activity in a sample from the patient indicates that the individual with Pompe disease is responding to chaperone treatment. In one non-limiting embodiment, the method of determining the activity of GAA activity in the sample is an assay comprising parallel enzymatic reactions, wherein a first enzymatic reaction is conducted in the absence of an antibody to GAA (anti-GAA antibodies), and a second enzymatic reaction is conducted in the presence of an antibody to GAA, wherein a difference between the two enzymatic assays indicates GAA activity.

### BRIEF DESCRIPTION OF THE DRAWINGS

FIG. 1 depicts peripheral blood lymphocytes isolated by Ficoll centrifugation.

FIG. 2 depicts α-glucosidase activity as a function of pH in lymphocytes and granulocytes derived from blood serum.

FIG. 3 depicts α-glucosidase activity in lysates from Ficoll-isolated lymphocytes in the presence of increasing amounts of anti-GAA antibody.

FIG. 4 depicts α-glucosidase activity in lysates from Ficoll-isolated lymphocytes from healthy volunteers in the presence and absence of anti-GAA antibody.

FIG. 5 depicts the precision of the anti-GAA antibodies based method of measuring GAA activity. GAA activity from three independent blood samples from a single healthy donor were determined using the anti-GAA based assay, demonstrating the repeatability of the method.

FIG. 6A-B depicts α-glucosidase activity measured in lysates from Ficoll-isolated lymphocytes with simulated low levels of GAA.

### DETAILED DESCRIPTION

Pompe disease is often difficult to diagnose in part because GAA enzyme activity measurements in blood or mixed leukocyte samples (lymphocytes with up to 10% contaminating granulocytes) are unreliable using the traditional 4-methylumbelliferyl-α-D-glucopyranoside (4MU-α-Glc) fluorometric assay. Multiple enzymes, predominantly maltase-glucoamylase (MGAM), hydrolyze this fluorogenic substrate and mask any GAA enzyme deficiency. The present invention is based on the discovery that the sensitivity and accuracy of assays which measure GAA activity in a sample can be improved by measuring GAA activity in parallel enzymatic reactions in the presence and absence of inhibitory anti-GAA antibodies. The activity from the sample without antibodies represents total α-glucosidase activity while the activity from the parallel sample containing antibodies is the non-GAA activity. The difference in activity between the two samples therefore represents GAA activity within a leukocyte preparation. This new method can be used to measure GAA activity, even at low GAA enzyme levels (<5% of WT) in reconstituted leukocyte lysates.

### DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Pompe disease” also referred to as acid maltase deficiency, glycogen storage disease type II (GSDII), and glycogenosis type II, is a genetic lysosomal storage disorder characterized by mutations in the GAA gene which metabolizes glycogen. As used herein, this term includes infantile, juvenile and adult-onset types of the disease.

Pompe disease is one of several of glycogen pathologies. Others include Debrancher deficiency (Cori's-Forbes' disease; Glycogenosis type III); Branching deficiency (Glycogenosis type IV; Andersen's disease); Myophsophorylase (McArdle's disease, Glycogen storage disease V); Phosphofructokinase deficiency-M isoform (Tauri's disease; Glycogenosis type VII); Phosphorylase b Kinase deficiency (Glycogenosis type VIII); Phosphoglycerate kinase A-isoform deficiency (Glycogenosis IX); Phosphoglycerate M-mutase deficiency (Glycogenosis type X).

As used herein, a “patient” or “subject” refers to an individual or animal who has been diagnosed with, or suspected of having, a particular disease. The patient may be human or animal. A “Pompe disease patient” refers to an individual who has been diagnosed with, or suspected of having, Pompe disease, and/or has a mutated GAA and/or reduced GAA activity, as defined and discussed further below.

As used herein, the terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence.

As used herein the term “mutant protein.” refers to proteins translated from genes containing genetic mutations that result in altered protein sequences. In a specific embodiment, such mutations result in the inability of the protein to achieve its native conformation under the conditions normally present in the ER. The failure to achieve this conformation results in these proteins being degraded, rather than being transported through their normal pathway in the protein transport system to their proper location within the cell. Other mutations can result in decreased activity or more rapid turnover.

As used herein the term “wild-type gene” refers to a nucleic acid sequences which encodes a protein capable of having normal biological functional activity in vivo. The wild-type nucleic acid sequence may contain nucleotide changes that differ from the known, published sequence, as long as the changes result in amino acid substitutions having little or no effect on the biological activity. The term wild-type may also include nucleic acid sequences engineered to encode a protein capable of increased or enhanced activity relative to the endogenous or native protein.

As used herein, the term “wild-type protein” refers to any protein encoded by a wild-type gene that is capable of having functional biological activity when expressed or introduced in vivo. The term “normal wild-type activity” refers to the normal physiological function of a protein in a cell. Such functionality can be tested by any means known to establish functionality of a protein.

As used herein the term “mutant α-glucosidase” or “mutant GAA” refers to an α-glucosidase polypeptide translated from a gene containing a genetic mutation that results in an altered α-glucosidase amino acid sequence. In one embodiment, the mutation results in an α-glucosidase protein that does not achieve a native conformation under the conditions normally present in the ER, when compared with wild-type α-glucosidase or exhibits decreased stability or activity as compared with wild-type α-glucosidase. This type of mutation is referred to herein as a “conformational mutation,” and the protein bearing such a mutation is referred as a “conformational mutant.” The failure to achieve this conformation results in the α-glucosidase protein being degraded or aggregated, rather than being transported through a normal pathway in the protein transport system to its native location in the cell or into the extracellular environment. In some embodiments, a mutation may occur in a non-coding part of the gene encoding α-glucosidase that results in less efficient expression of the protein, e.g., a mutation that affects transcription efficiency, splicing efficiency, mRNA stability, and the like. By enhancing the level of expression of wild-type as well as conformational mutant variants of α-glucosidase, administration of an α-glucosidase pharmacological chaperone can ameliorate a deficit resulting from such inefficient protein expression. Alternatively, for splicing mutants or nonsense mutants which may accumulate in the ER, the ability of the chaperone to bind to and assist the mutants in exiting the ER, without restoring lysosomal hydrolase activity, may be sufficient to ameliorate some cellular pathologies in Pompe patients, thereby improving symptoms.

Exemplary conformational mutations of GAA include the following: D645E (Lin et al., Zhonghua Min Guo Xiao Er Ke Yi Xue Hui Za Zhi. 1996; 37(2):115-21); D645H (Lin et al., Biochem Biophys Res Commun. 1995 17; 208(2):886-93); R224W, S619R, and R660H (New et al. Pediatr Neurol. 2003; 29(4):284-7); T1064C and C2104T (Montalvo et al., Mol Genet Metab. 2004; 81(3):203-8); D645N and L901Q (Kroos et al., Neuromuscul Disord. 2004; 14(6):371-4); G219R, E262K, M408V (Fernandez-Hojas et al., Neuromuscul Disord. 2002; 12(2):159-66); G309R (Kroos et al., Clin Genet. 1998; 53(5):379-82); D645N, G448S, R672W, and R672Q (Huie et al., Biochem Biophys Res Commun. 1998; 27; 244(3):921-7); P545L (Hermans et al., Hum Mol Genet. 1994; 3(12):2213-8); C647W (Huie et al., Huie et al, Hum Mol Genet. 1994; 3(7):1081-7); G643R (Hermans et al., Hum Mutat, 1993; 2(4):268-73); M318T (Zhong et al., Am J Hum Genet. 1991; 49(3):635-45); E521K (Hermans et al., Biochem Biophys Res Commun. 1991; 179(2):919-26); W481R (Raben et al., Hum Mutat. 1999; 13(1):83-4); and L552P and G549R (unpublished data).

Splicing mutants of GAA include IVS1AS, T>G, −13 and IVS8+1G>A.

Additional GAA mutants have been identified and are known in the art.

As used herein, the terms “enhance GAA activity” or “increase GAA activity” refer to increasing the amount of GAA that adopts a stable conformation in a cell contacted with a pharmacological chaperone specific for the GAA, relative to the amount in a cell (preferably of the same cell-type or the same cell, e.g., at an earlier time) not contacted with the pharmacological chaperone specific for the GAA. This term also refers to increasing the trafficking of GAA to the lysosome in a cell contacted with a pharmacological chaperone specific for the GAA, relative to the trafficking of GAA not contacted with the pharmacological chaperone specific for the protein. These terms refer to both wild-type and mutant GAA. In one embodiment, the increase in the amount of GAA in the cell is measured by measuring the hydrolysis of an artificial substrate in lysates from cells that have been treated with the SPC, described further herein below. An increase in hydrolysis is indicative of increased GAA activity.

The term “enzyme replacement therapy” or “ERT” refer to the introduction of a non-native, purified enzyme into an individual having a deficiency in such enzyme. The administered enzyme can be obtained from natural sources or by recombinant expression. The term also refers to the introduction of a purified enzyme in an individual otherwise requiring or benefiting from administration of a purified enzyme, e.g., suffering from protein insufficiency. GAA may be obtained from commercial sources or may be obtained by synthesis techniques known to a person of ordinary skill in the art. The wild-type enzyme can be purified from a recombinant cellular expression system (e.g., mammalian cells or insect cells-see generally U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al.; and U.S. Pat. No. 5,879,680 to Ginns et al.), human placenta, or animal milk (see U.S. Pat. No. 6,188,045 to Reuser et al.), or from plants. After the infusion, the exogenous enzyme is expected to be taken up by tissues through non-specific or receptor-specific mechanism. In general, the uptake efficiency (without use of an ASSC) is not high, and the circulation time of the exogenous protein is short (Ioannu et al., Am. J. Hum. Genet. 2001; 68: 14-25). In addition, the exogenous protein is unstable and subject to rapid intracellular degradation in vitro.

Other synthesis techniques for obtaining GAA suitable for pharmaceutical may be found, for example, in U.S. Pat. Nos. 7,423,135, 6,534,300, and 6,537,785; International Published Application No. 2005/077093 and U.S. Published Application Nos. 2007/0280925, and 2004/0029779. These references are hereby incorporated by reference in their entirety.

In one embodiment, the GAA is a recombinant human GAA (rhGAA), for example, GAA is alglucosidase alfa, which consists of the human enzyme acid α-glucosidase (GAA), encoded by the most predominant of nine observed haplotypes of this gene and is produced by recombinant DNA technology in a Chinese hamster ovary cell line. Alglucosidase alpha is available as Myozyme®, from Genzyme Corporation (Cambridge, Mass.). A recombinant GAA may be produced and used in ERT, for example, according to the methods described in U.S. Pat. No. 7,056,712; and U.S. Published Application Nos. 2005/123531 and 20081175833; each of which are incorporated by reference in their entireties herein for all purposes.

In one embodiment, a subject is administered a combination of a specific pharmacological chaperone (e.g. a reversible competitive inhibitor (e.g. 1-DNJ)) and ERT (e.g. a recombinant GAA, rhGAA, (e.g., Myozyme©, Genzyme Corp., Cambridge, Mass.)).

As used herein, the term “specific pharmacological chaperone” (“SPC”) refers to any molecule including a small molecule, protein, peptide, nucleic acid, carbohydrate, etc. that specifically binds to a protein and has one or more of the following effects: (i) enhancing the formation of a stable molecular conformation of the protein; (ii) inducing trafficking of the protein from the ER to another cellular location, preferably a native cellular location, i.e., preventing ER-associated degradation of the protein; (iii) preventing aggregation of misfolded proteins; and/or (iv) restoring or enhancing at least partial wild-type function and/or activity to the protein. A compound that specifically binds to e.g., GAA, means that it binds to and exerts a chaperone effect on GAA and not a generic group of related or unrelated enzymes.

In one embodiment, the SPC is a competitive inhibitor of GAA, A “competitive inhibitor” of an enzyme can refer to a compound which structurally resembles the chemical structure and molecular geometry of the enzyme substrate to bind the enzyme in approximately the same location as the substrate. Thus, the inhibitor competes for the same active site as the substrate molecule, thus increasing the Km. Competitive inhibition is usually reversible if sufficient substrate molecules are available to displace the inhibitor, i.e., competitive inhibitors can bind reversibly. Therefore, the amount of enzyme inhibition depends upon the inhibitor concentration, substrate concentration, and the relative affinities of the inhibitor and substrate for the active site.

The term “stabilize a proper conformation” refers to the ability of a compound or peptide or other molecule to associate with a wild-type protein, or to a mutant protein that can perform its wild-type function in vitro and in vivo, in such a way that the structure of the wild-type or mutant protein can be maintained as its native or proper form. This effect may manifest itself practically through one or more of (i) increased shelf-life of the protein; (ii) higher activity per unit/amount of protein; or (iii) greater in viva efficacy. It may be observed experimentally through increased yield from the ER during expression; greater resistance to unfolding due to temperature increases (e.g. as determined in thermal stability assays), or the present of chaotropic agents, and by similar means.

Following is a description of some specific pharmacological chaperones contemplated by this invention:

1-deoxynojirimycin (DNJ) refers to a compound having the following structures:

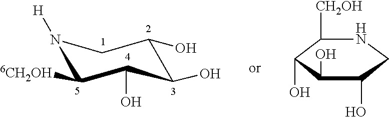

This term includes both the free base and any salt forms.

Still other SPCs for GAA are described in U.S. Pat. No. 6,599,919 to Fan et al., and U.S. Patent Application Publication US 20060264467 to Mugrage et al., both of which are herein incorporated by reference in their entireties, and include N-methyl-DNJ, N-ethyl-DNJ, N-propyl-DNJ, N-butyl-DNJ, N-pentyl-DNJ, N-hexyl-DNJ, N-heptyl-DNJ, N-octyl-DNJ, N-nonyl-DNJ, N-methylcyclopropyl-DNJ, N-methylcyclopentyl-DNJ, N-2-hydroxyethyl-DNJ, and 5-N-carboxypentyl DNJ.

A “responder” is an individual diagnosed with a disease associated with a GAA mutation which causes misfolding of the GAA protein, such as Pompe disease, and treated with SPC therapy, ERT or who exhibits an improvement in, amelioration of, or prevention of, one or more clinical symptoms, or whose GAA activity increases following treatment.

The terms “therapeutically effective dose” and “effective amount” refer to the amount of the specific pharmacological chaperone that is sufficient to result in a therapeutic response. A therapeutic response may be any response that a user (e.g., a clinician) will recognize as an effective response to the therapy, including improvements in the foregoing symptoms and increases in the patient's GAA activity following treatment. Thus, a therapeutic response will generally be an amelioration of one or more symptoms of a disease or disorder, such as those described above.

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

### Diagnosing and Monitoring Treatment of Pompe Disease Using a GAA Activity Assay

The present invention provides a method for diagnosing Pompe disease in an individual by detecting decreased levels of GAA activity in a sample from the patient compared to a sample from an individual who does not have the disease. The present invention also provides a method for monitoring the treatment of Pompe patients with specific pharmacological chaperones, ERT, or both. In one embodiment, the patient is treated with the specific pharmaceutical chaperone DNJ.

Decreased GAA is associated with Pompe disease. Assessment of GAA activity can be evaluated in a sample from a patient, for example, in peripherally obtained lymphoblasts, leukocytes and polymorphonuclear cells (PMNs) derived from Pompe patients. Cultured fibroblasts from skin biopsies can also be used. Such assays typically involve extraction of blood leukocytes from the patient, lysing the cells, and determining the activity upon addition of a substrate such as 4-methyl umbeliferryl-α-D-glucopyranoside (4MU-α-Glc) (see e.g., Hermans et al. Human Mutation 2004; 23: 47-56).

The present invention provides advantages over other GAA activity assays, for example, assays that determine GAA activity by measuring the hydrolysis of 4MU-α-Glc, wherein the activity of multiple enzymes, for example, maltase-glucoamylase (MGAM), mask the activity of GAA, and as such, deficiencies in GAA activity can not be detected.

Specifically, the method employs an assay to diagnose Pompe disease, and/or to evaluate the progress of the disease and its response to treatment, wherein the GAA activity in a sample from the patient can be assayed, for example, by measuring the hydrolysis of a GAA substrate. In one embodiment, the hydrolysis of the substrate is measured in the absence of an anti-GAA antibody. In a further embodiment, GAA activity is measured in a parallel sample from the same patient, wherein GAA activity is measured in the presence of an anti-GAA antibody. The activity of GAA can then be determined as the difference between the enzymatic activity measured in the absence of the anti-GAA antibody and the enzymatic activity measured in the presence of the anti-GAA antibody.

In one embodiment, Pompe disease can be diagnosed in an individual by comparing the GAA activity level in a sample from the individual to the GAA activity measured in a second sample, wherein the second sample is derived from a healthy volunteer who does not have Pompe disease. A decreased level of GAA activity in the sample from the individual compared to the level of GAA activity in the sample from the healthy volunteer indicates that the individual has Pompe disease.

In other non-limiting embodiments, the methods of the present example can be used to monitor the progress of treatment of a Pompe patient. In certain embodiments, the level of GAA activity can be measured in a sample from the patient prior to initiation of treatment. GAA activity can then be measured in a second sample taken from the patient following initiation of the Pompe treatment. An increase in GAA in the second sample compared to the GAA activity in the first sample indicates that the patient is responding to the Pompe treatment.

In one non-limiting embodiment, the methods of the present invention can detect and measure GAA enzyme activity levels in a reconstituted leukocyte lysate sample, wherein the GAA activity level is from at least about 0-0.01% of wild type, from at least about 0.01-0.1% of wild type, from at least about 0.1-0.5% of wild type, from at least about 0.5-1% of wild type, or from at least about 1-5% or more of wild type.

In one non-limiting embodiment, the anti-GAA antibody is a polyclonal antibody.

In other non-limiting embodiments, the anti-GAA antibody is a monoclonal antibody.

Flow cytometry can also be used to evaluate GAA activity in patient cells (Lorincz et al., Blood. 1997; 189: 3412-20; and Chan et al., Anal Biochem. 2004; 334(2):227-33). This method employs a fluorogenic GAA substrate which can be loaded into cells by pinocytosis. The cells are then evaluated using conventional fluorescein emission optics. Levels of fluorescence correlate with the amount of GAA activity.

### Formulations, Dosage, and Administration

DNJ and derivatives can be administered in a form suitable for any route of administration, including e.g., orally in the form tablets, capsules, or liquid, or in sterile aqueous solution for injection. In a specific embodiment, the DNJ (e.g. DNJ hydrochloride) is administered as a powder-filled capsule. When the compound is formulated for oral administration, the tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art.

Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., water, sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled or sustained release of the ceramide-specific glucosyltransferase inhibitor.

The pharmaceutical formulations of DNJ or derivatives suitable for parenteral/injectable use generally include sterile aqueous solutions, or dispersions and sterile powders for the extemporaneous preparation of sterile injectable solutions or dispersion. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate and gelatin.

Sterile injectable solutions are prepared by incorporating DNJ or derivatives in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The above formulations can contain an excipient or excipients. Pharmaceutically acceptable excipients which may be included in the formulation are buffers such as citrate buffer, phosphate buffer, acetate buffer, and bicarbonate buffer, amino acids, urea, alcohols, ascorbic acid, phospholipids, proteins, such as serum albumin, collagen, and gelatin; salts such as EDTA or EGTA, and sodium chloride; liposomes; polyvinylpyrollidone; sugars such as dextran, mannitol, sorbitol, and glycerol; propylene glycol and polyethylene glycol (e.g., PEG-4000, PEG-6000); glycerol, glycine or other amino acids and lipids. Buffer systems for use with the formulations include citrate, acetate, bicarbonate, and phosphate buffers. Phosphate buffer is a preferred embodiment.

The formulations can also contain a non-ionic detergent. Preferred non-ionic detergents include Polysorbate 20, Polysorbate 80, Triton X-100, Triton X-114, Nonidet P-40, Octyl α-glucoside, Octyl β-glucoside, Brij 35, Pluronic, and Tween 20.

### Administration

The route of administration of DNJ or derivatives may be oral (preferably) or parenteral, including intravenous, subcutaneous, intra-arterial, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, or via inhalation.

Administration of the above-described parenteral formulations of DNJ or derivatives may be by periodic injections of a bolus of the preparation, or may be administered by intravenous or intraperitoneal administration from a reservoir which is external (e.g., an i.v. bag) or internal (e.g., a bioerodable implant). See, e.g., U.S. Pat. Nos. 4,407,957 and 5,798,113, each incorporated herein by reference. Intrapulmonary delivery methods and apparatus are described, for example, in U.S. Pat. Nos. 5,654,007, 5,780,014, and 5,814,607, each incorporated herein by reference. Other useful parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aeorosolizer, electroporation, and transdermal patch. Needle-less injector devices are described in U.S. Pat. Nos. 5,879,327; 5,520,639; 5,846,233 and 5,704,911, the specifications of which are herein incorporated by reference. Any of the formulations described above can be administered using these methods.

Furthermore, a variety of devices designed for patient convenience, such as refillable injection pens and needle-less injection devices, may be used with the formulations of the present invention as discussed herein.

### Dosage

Persons skilled in the art will understand that an effective amount of the DNJ or derivatives used in the methods of the invention can be determined by routine experimentation. As a non-limiting example, the doses and regimens expected to be sufficient to increase GAA in most “rescuable” individuals is as described in U.S. Provisional Application 61/028,105, filed Feb. 12, 2008, herein incorporated by reference in its entirety.

### EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### Example 1

### Ficoll Density Gradient Isolation Methods Reduce but do not Eliminate Contaminating Neutrophil MGAM Activity from Lymphocyte Preparations

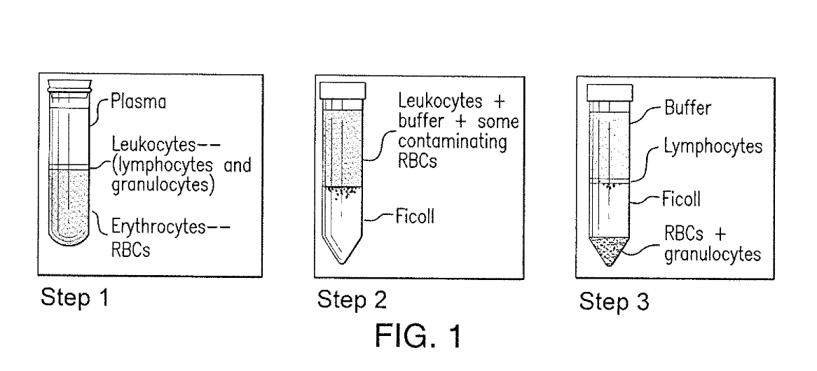

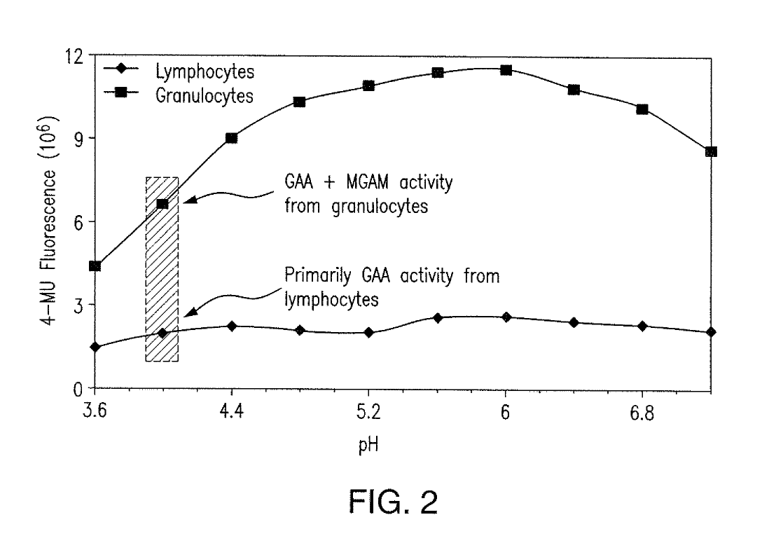

In order to identify individuals with Pompe disease, methods to measure GAA activity in blood samples have been developed. These methods include measuring the hydrolysis of the substrate 4MU-α-Glc by GAA. Such methods identify individuals with Pompe disease by detecting a decreased level of GAA activity in samples from the individuals compared to GAA activity in samples from individuals without the disease. However, these methods are not suitable to accurately measure small differences in GAA activity, primarily due to contaminating neutrophil granulocytes which express maltase glucoamylase (MGAM). The hydrolysis of the GAA substrate 4MU-α-Glc at pH 4 by MGAM masks low levels of GAA activity (FIG. 2). Cell isolation methods using density gradients (e.g., Ficoll) (FIG. 1) enrich for GAA expressing lymphocytes, but do not completely remove granulocytes. Additional enrichment via immunomagnetic sorting or inhibition with acarbose helps to reduce the contribution from MGAM activity but does not eliminate the contaminating α-glucosidase activity.

### Example 2

### Distinguishing GAA Activity from Contaminating α-Glucosidases with Anti-GAA Antibodies

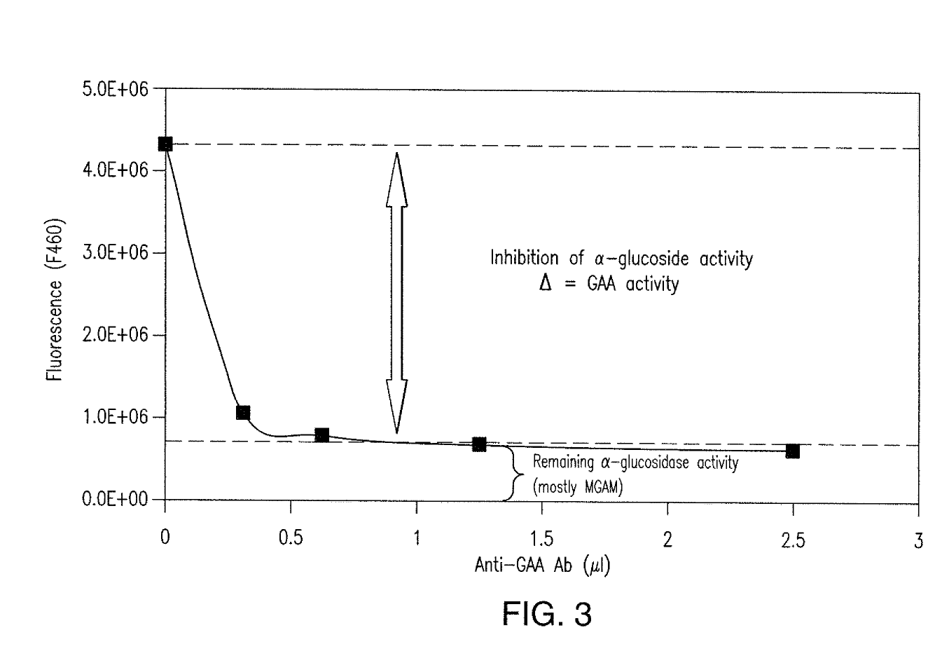

α-Glucosidase activity was measured in lysates from Ficoll-isolated lymphocytes. GAA enzymatic activity was measured as a function of the enzyme's ability to hydrolyse the GAA substrate 4MU-α-Glc. Fluorescence of the hydrolyzed substrates indicates GAA enzymatic activity. Polyclonal anti-GAA antibodies specifically bind to GAA and inhibit enzyme activity. Enzymatic reactions (using 4MU-α-Glc) were performed in parallel with or without anti-GAA Ab. The amount of GAA activity is determined by the difference between these samples (i.e., Δ=GAA activity) (FIG. 3).

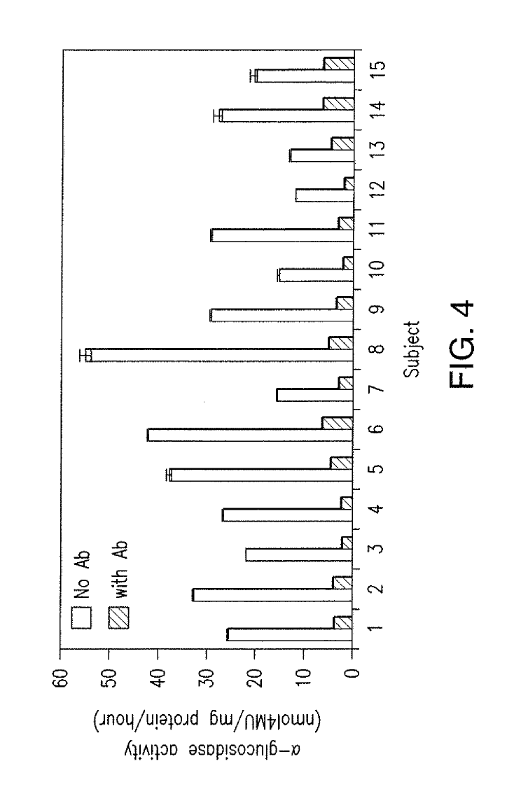

GAA activity was measured in lysates from Ficoll-isolated lymphocytes from 15 healthy volunteers in parallel assays. A first assay was conducted in the presence of anti-GAA antibody, while a second assay was conducted in the absence of the antibody. The difference between the two assays is the enzymatic activity due to GAA. The observed range of GAA activity in normal healthy donors was from 10-50 nmol 4MU/mg/hr, with an average GAA activity of 23.2±2.8 nmol (FIG. 4). Such results are consistent with previously published reports (Okumiya et al., 2005).

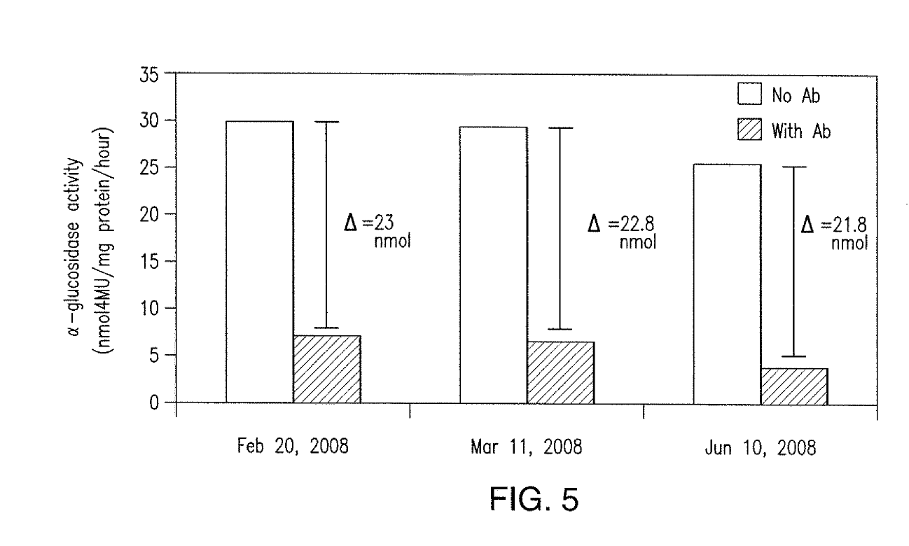

GAA activity was independently measured in lymphocytes isolated from a single healthy donor on three separate occasions (Feb. 20, 2008; Mar. 11, 2008 and Jun. 10, 2008). The average GAA activity for the three samples was 22.5±0.3 nmol 4MU/mg protein/hr (FIG. 5). Such results demonstrates the reliability of the assay utilizing the anti-GAA antibodies.

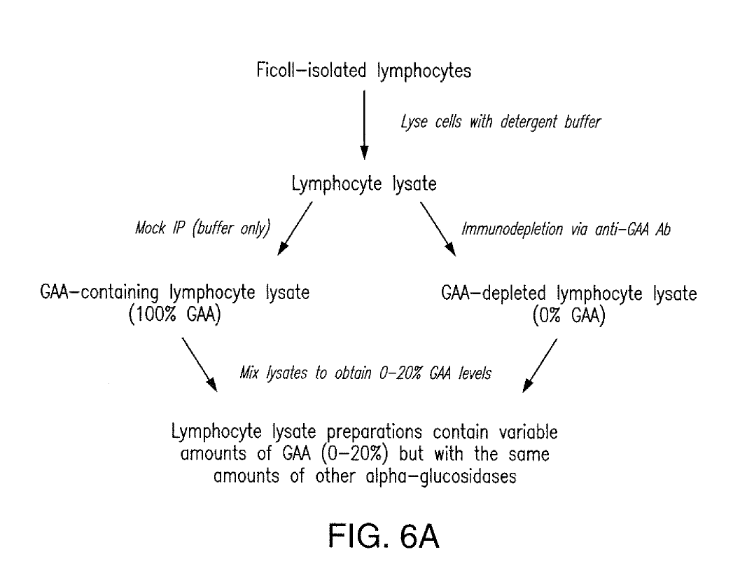

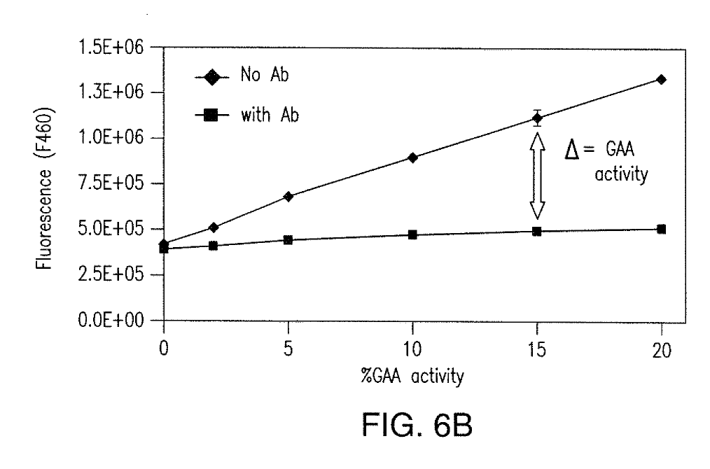

Furthermore, the use of anti-GAA antibodies in the assays to measure GAA activity can detect small differences in GAA activity between samples. α-Glucosidase activity was measured in Ficoll-isolated lymphocyte lysates with simulated low levels of GAA. As shown in FIG. 6A, a series of low level GAA samples were created from lysates from Ficoll-isolated lymphocytes. The lysates were divided into two samples, wherein anti-GAA antibodies were added to one sample, inhibiting GAA activity (0% GAA). In the parallel sample, no anti-GAA antibodies were added (100% GAA). The two lysates were mixed in various amounts to create the series of lysates which contained from 0-20% GAA activity, but which retained the same level of enzymatic activity from other non-GAA α-glucosidases (e.g., MGAM). The GAA activity of the series was then measured in the presence and absence of anti-GAA antibodies. As shown in FIG. 6B, the level of non-GAA glucosidase activity remained constant, while the increase in activity due to GAA was detectable as the level of GAA increased from 0-20% in the series.

Measuring GAA activity in peripheral lymphocyte samples is unreliable due to contaminating α-glucosidases, predominantly MGAM. The method described herein is an adaptation of the traditional 4MU-α-Glc fluorometric GAA activity assay, but utilizes anti-GAA antibodies to distinguish between GAA-specific activity and activity from other α-glucosidases in peripheral lymphocyte preparations (e.g., MGAM). This assay can accurately measure relatively low GAA levels (<5% of WT) in cell lysates, and as such, this improved method has multiple applications, as a useful research tool and as the basis of a clinical assays designed to accurately measure GAA activity in patient samples for the diagnosis of Pompe disease. This assay is also useful to measure GAA activity in Pompe patients undergoing SPC treatment, ERT treatment, or both, in order to monitor the progression and success of the treatment.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for determining acid α-glucosidase activity in a sample from an individual comprising:
(a) measuring acid α-glucosidase enzymatic activity of the sample in the absence of an anti-GAA antibody;
(b) measuring acid α-glucosidase enzymatic activity of the sample in the presence of an anti-GAA antibody; and
(c) comparing the acid α-glucosidase enzymatic activities from steps (a) and (b), wherein a difference in enzymatic activity between (a) and (b) is the acid α-glucosidase activity in the sample.

2. The method of claim 1 further comprising the step of comparing the acid α-glucosidase activity in the sample to the acid α-glucosidase activity in a second sample from an individual who does not have Pompe disease, wherein a decreased level of acid α-glucosidase activity in the first sample compared to the second sample indicates Pompe disease.

3. The method of claim 1 further comprising the step of comparing the acid α-glucosidase activity in the sample to the acid α-glucosidase activity in a second sample from a healthy individual, wherein a decreased level of acid α-glucosidase activity in the first sample compared to the second sample indicates the presence of a disorder in the first individual selected from the group consisting of Debrancher deficiency (Cori's-Forbes' disease; Glycogenosis type III); Branching deficiency (Glycogenosis type IV; Andersen's disease); Myophsophorylase (McArdle's disease, Glycogen storage disease V); Phosphofructokinase deficiency-M isoform (Tauri's disease; Glycogenosis type VII); Phosphorylase b Kinase deficiency (Glycogenosis type VIII); Phosphoglycerate kinase A-isoform deficiency (Glycogenosis IX); and Phosphoglycerate M-mutase deficiency (Glycogenosis type X).

4. The method of claim 1, wherein an acid α-glucosidase substrate hydrolysis assay is used to measure acid α-glucosidase enzymatic activity.

5. The method of claim 4, wherein the substrate is 4-methylumbelliferyl-α-D-glucopyranoside.

6. The method of claim 1, wherein the sample is selected from the group consisting of lymphoblasts, leukocytes, polymorphonuclear cells (PMNs) and fibroblasts.

7. The method of claim 1, wherein the anti-GAA antibody is a polyclonal antibody.

8. The method of claim 1, wherein the anti-GAA antibody is a monoclonal antibody.

9. A method for monitoring a therapeutic response of a Pompe disease patient following administration of an amount of a specific pharmacological chaperone of acid α-glucosidase A, which method comprises determining whether there is an increase in acid α-glucosidase activity in a sample from the patient.

10. The method of claim 9, wherein the acid α-glucosidase activity in the sample is determined according to an assay comprising:
(a) measuring acid α-glucosidase enzymatic activity of the sample in the absence of an anti-GAA antibody;
(b) measuring acid α-glucosidase enzymatic activity of the sample in the presence of an anti-GAA antibody;
(c) comparing the acid α-glucosidase enzymatic activities from steps (a) and (b), wherein a difference in enzymatic activity between (a) and (b) is the acid α-glucosidase activity in the sample; and
(d) comparing the acid α-glucosidase activity from (c) with an acid α-glucosidase activity measured in a sample from the patient prior to the administration of the specific pharmacological chaperone to the patient, wherein a greater acid α-glucosidase activity in (c) compared to the acid α-glucosidase activity measured in the sample from the patient prior to the administration of the specific pharmacological chaperone indicates a positive therapeutic response.

11. The method of claim 9, wherein the specific pharmacological chaperone is an inhibitor of α-glucosidase A.

12. The method of claim 11, wherein the inhibitor is a reversible competitive inhibitor.

13. The method of claim 12, wherein the inhibitor is 1-deoxynojirimycin.

14. The method of claim 9, wherein an acid α-glucosidase substrate hydrolysis assay is used to measure acid α-glucosidase enzymatic activity.

15. The method of claim 14, wherein the substrate is 4-methylumbelliferyl-α-D-glucopyranoside.

16. The method of claim 9, wherein the sample is selected from the group consisting of lymphoblasts, leukocytes, polymorphonuclear cells (PMNs) and fibroblasts.

17. The method of claim 10, wherein the anti-GAA antibody is a polyclonal antibody.

18. The method of claim 10, wherein the anti-GAA antibody is a monoclonal antibody.
